(19) United States
(12) Patent Application Publication
(10) Pub. No.: US 2023/0148035 A1
(43) Pub. Date: May 11, 2023

(54) CLEAN ACCESS UV

(71) Applicants: John Ayala, claremont, CA (US); Kevin Escoto, Claremont, CA (US)

(72) Inventors: John Ayala, Claremont, CA (US); Kevin Escoto, Claremont, CA (US)

(21) Appl. No.: 17/521,511

(22) Filed: Nov. 8, 2021

(51) Int. Cl. A61L 2/10

(52) U.S. Cl. CPC A61L 2/10; A61L 2202/11; A61L 2202/122; A61L 2202/14

(57) ABSTRACT

An ultraviolet germicidal irradiation device includes a housing having one or two apertures where an access key card may be inserted and withdrawn. A conveyor operates to receive the card through one aperture, planarly move it along an axial direction of the housing, and eject the card through the same or a second aperture located at the opposite end of the housing. An ultraviolet light source illuminates the card as the card moves along the housing, thereby, disinfecting the card. A control device is configured to control a linear motion of the conveyor along the axial direction of the housing.

### COPYRIGHT

A portion of the disclosure of this patent document contains material which is subject to copyright protection. The owner has no objection to the facsimile reproduction by anyone of the patent disclosure, as it appears in the Patent and Trademark Office files or records, but otherwise reserves all copyright rights whatsoever.

### FIELD OF INVENTION

The present invention relates to an ultraviolet germicidal irradiation device and a method of disinfecting a card via the ultraviolet germicidal irradiation device. The device may be used to disinfect a card, such as a hotel key card, using ultraviolet light sources. A housing and a conveyor mechanism within the housing moves the card in a planarly manner while the card is illuminated via the ultraviolet light sources. A control device controls the linear motion of the conveyor along the axial direction of the housing.

### BACKGROUND

Guest management systems in hotels use access key cards to allow guests to open their assigned rooms. The card is handled by a large number of guests and as such present a health issue. In particular, each guest carries numerus germs on his body and in particular on his hands. Accordingly, those germs will be deposited on the card and subsequently transmitted to other guests.

Ultraviolet light sources in the UVC band have been used extensively for disinfection of surfaces in laboratories and food-processing industries. For instance, U.S. Pat. No. 9,603,956 discloses a system for disinfecting a clinical surface utilizing a broad-spectrum UV-C ultraviolet light source, U.S. Pat. No. 9,974,875 discloses a sterilizer for point-of-sale credit card readers and keypads using a UV-C ultraviolet light source, and U.S. Pat. No. 11,097,025 discloses a system and method for sterilizing gaming machines and associated equipment.

The present invention is an ultraviolet germicidal irradiation device which includes a housing having one or two apertures where a key card maybe inserted, disinfected via an ultraviolet light source, and ejected free of germs. A conveyor within the housing operates to receive the card and planarly move it along an axial direction of the housing. An ultraviolet light source illuminates the card while it moves along the housing. A control device is configured to control a linear motion of the conveyor, for instance, its speed, to ensure that the ultraviolet light source properly illuminates the card thereby disinfecting the card as it moves along the housing.

### SUMMARY

In one aspect, an ultraviolet germicidal irradiation device is disclosed wherein the devices comprises a substantially cubic housing having a first aperture through a first side of the housing and a second aperture through a second side of the housing, a conveyor operable to receive a card through the first aperture, to planarly move the card along an axial direction of the housing, and to eject the card through the second aperture, an ultraviolet light source operable to disinfect the card, wherein the ultraviolet light source illuminates the card within the housing as the card moves along the axial direction of the housing, and a control device, coupled with the conveyor, wherein the control device comprises a processor including a programming code stored on a storage device of said processor, and wherein the control device is configured to control a linear motion of the conveyor along the axial direction of the housing.

Preferably, each of the first aperture and the second aperture has a rectangular cross section.

Preferably, a width of the rectangular cross section of each of the first aperture and the second aperture is substantially equal to or greater than a width of a rectangular cross section of the card, and wherein a height of the rectangular cross section of each of the first aperture and the second aperture is substantially equal to or greater than a height of the rectangular cross section of the card.

Preferably, the conveyor comprises a first roller, a second roller, a third roller, a fourth roller, a first belt, and a motor coupled with the control device, wherein the first belt engages a first end of the second roller and a first end of the third roller, and wherein the motor engages the second roller.

Preferably, the conveyor further comprises a second belt operable to engage a second end of the second roller and a second end of the third roller.

Preferably, the conveyor comprises a first roller having a first gear, a second roller having a second gear, a third roller having a third gear, a fourth roller having a fourth gear, a first belt, and a motor coupled with the control device, wherein the first gear engages the second gear, wherein the third gear engages the fourth gear, wherein the first belt engages the second roller and the third roller, and wherein the motor engages the first roller.

Preferably, the conveyor further comprises a second belt operable to engage the first roller and the fourth roller.

Preferably, the ultraviolet light source comprises four panels having UV-C light emitting diodes (LEDs).

Preferably, the four panels illuminate the card from a top side, a bottom side, a left side, and a right side of the card.

Preferably, the control device is further coupled with ultraviolet light source and configured to at least one of turn on and turn off the ultraviolet light source.

Preferably, the device further comprises a sensor operable to detect the card through the first aperture and to generate a signal in response to detecting the card, wherein the control device is further coupled with the sensor and configured to receive the signal from the sensor and control the linear motion of the conveyor along the axial direction of the housing according to the signal.

Preferably, the card is one of a hotel key card, an ATM card, and a credit card.

In another aspect, an ultraviolet germicidal irradiation device is disclosed wherein the device comprises a substantially cubic housing having an, aperture through a side of the housing, a conveyor operable to receive a card through the aperture, to planarly move the card along an axial direction of the housing, and to eject the card through the aperture, an ultraviolet light source operable to disinfect the card, wherein the ultraviolet light source illuminates the card within the housing as the card moves along the axial direction of the housing, and a control device, coupled with the conveyor, wherein the control device comprises a processor including a programming code stored on a storage device of said processor, and wherein the control device is configured to control a linear motion of the conveyor along the axial direction of the housing.

Preferably, the aperture has a rectangular cross section.

Preferably, a width of the rectangular cross section of the aperture is substantially equal to or greater than a width of a rectangular cross section of the card, and wherein a height of the rectangular cross section of the aperture is substantially equal to or greater than a height of the rectangular cross section of the card.

Preferably, the conveyor comprises a first roller, a second roller, a third roller, a fourth roller, a first belt, and a motor coupled with the control device, wherein the first belt engages a first end of the second roller and a first end of the third roller, and wherein the motor engages the second roller.

Preferably, the conveyor further comprises a second belt operable to engage a second end of the second roller and a second end of the third roller.

Preferably, the conveyor comprises a first roller having a first gear, a second roller having a second gear, a third roller having a third gear, a fourth roller having a fourth gear, a first belt, and a motor coupled with the control device, wherein the first gear engages the second gear, wherein the third gear engages the fourth gear, wherein the first belt engages the second roller and the third roller, and wherein the motor engages the first roller.

Preferably, the conveyor further comprises a second belt operable to engage the first roller and the fourth roller.

Preferably, the ultraviolet light source comprises four panels having UV-C light emitting diodes (LEDs).

Preferably, the four panels illuminate the card from a top side, a bottom side, a left side, and a right side of the card.

Preferably, the control device is further coupled with ultraviolet light source and configured to at least one of turn on and turn off the ultraviolet light source.

Preferably, the device further comprises a sensor operable to detect the card through the aperture and to generate a signal in response to detecting the card, wherein the control device is further coupled with the sensor and configured to receive the signal from the sensor and control the linear motion of the conveyor along the axial direction of the housing according to the signal.

In another aspect, a method of disinfecting a card via an ultraviolet germicidal irradiation device is disclosed wherein said ultraviolet germicidal irradiation device comprises a substantially cubic housing having a first aperture through a first side of the housing and a second aperture through a second side of the housing, a conveyor operable to receive the card through the first aperture, to planarly move the card along an axial direction of the housing, and to eject the card through the second aperture, an, ultraviolet light source operable to disinfect the card, wherein the ultraviolet light source illuminates the card within the housing as the card moves along the axial direction of the housing, and a control device, coupled with the conveyor, wherein the control device comprises a processor including a programming code stored on a storage device of said processor, and wherein the control device is configured to control a linear motion of the conveyor along the axial direction of the housing, wherein the method comprises receiving the card through the first aperture, via the conveyor, planarly moving the card along the axial direction of the housing, via the conveyor, illuminating the card within the housing as the card moves along the axial direction of the housing, via the ultraviolet light source, and controlling the linear motion of the conveyor along the axial direction of the housing, via the control device.

Preferably, the ultraviolet germicidal irradiation device further comprises a sensor operable to detect the card through the aperture and to generate a signal in response to detecting the card and wherein the control device is further coupled with the sensor and configured to receive the signal from the sensor and control the linear motion of the conveyor along the axial direction of the housing according to the signal, wherein the method further comprises detecting the card through the aperture, via the sensor, generating a signal in response to detecting the card, via the sensor, receiving the signal, via the control device, and controlling the linear motion of the conveyor along the axial direction of the housing according to the signal, via the control device.

### DESCRIPTION OF THE DRAWINGS

FIG. 1A shows a perspective view of an ultraviolet germicidal irradiation device according to a preferred embodiment. The device includes a housing having a first aperture through a first side of the housing.

FIG. 1B shows a perspective view of the ultraviolet germicidal irradiation device of FIG. 1A rotated 90 degrees clockwise around the Z axis. The housing has a second aperture through a second side of the housing.

FIG. 2 shows a perspective view of an ultraviolet germicidal irradiation device according to a preferred embodiment. The device includes a housing, a conveyor, an ultraviolet light source, and a control device.

FIG. 3 shows an exploded view of an ultraviolet light source according to a preferred embodiment. The ultraviolet light source comprises four panels having UV-C light emitting diodes (LEDs). A card planarly movies along an axial direction of a housing containing the ultraviolet light source wherein the card is disinfected by the radiation emanating from the UV-C ultraviolet light source as it moves along the axial direction.

FIG. 4 shows a perspective view of a conveyor and a control device according to a preferred embodiment. The conveyor includes a first roller, a second roller, a third roller, a fourth roller, a first belt, a second belt and a motor coupled with the control device. The first belt engages a first end of the second roller and a first end of the third roller. The motor engages the second roller. The second belt engages a second end of the second roller and a second end of the third roller.

### DETAILED DESCRIPTION OF THE PRESENTLY PREFERRED EMBODIMENTS

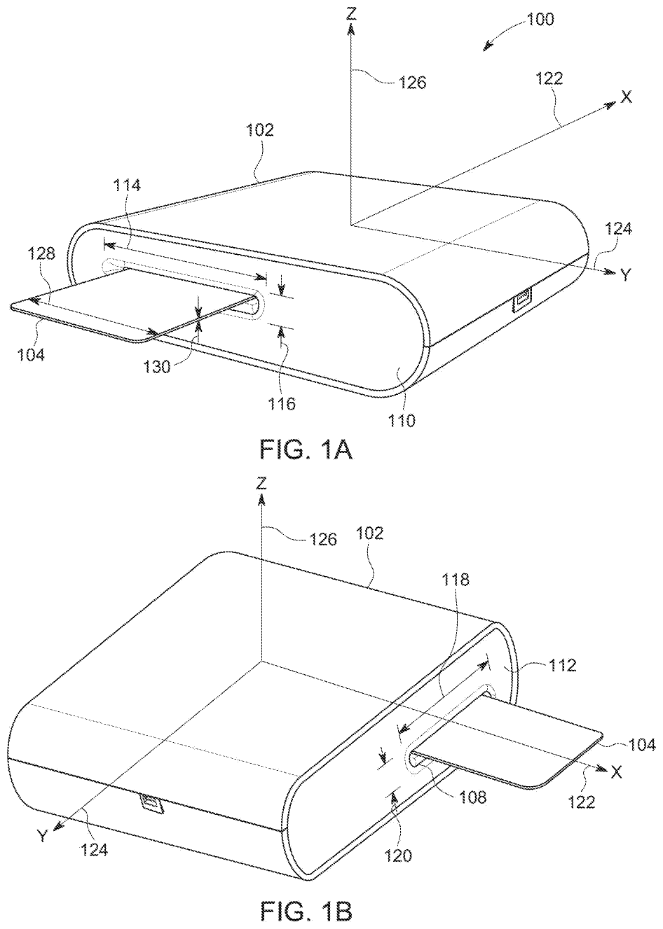

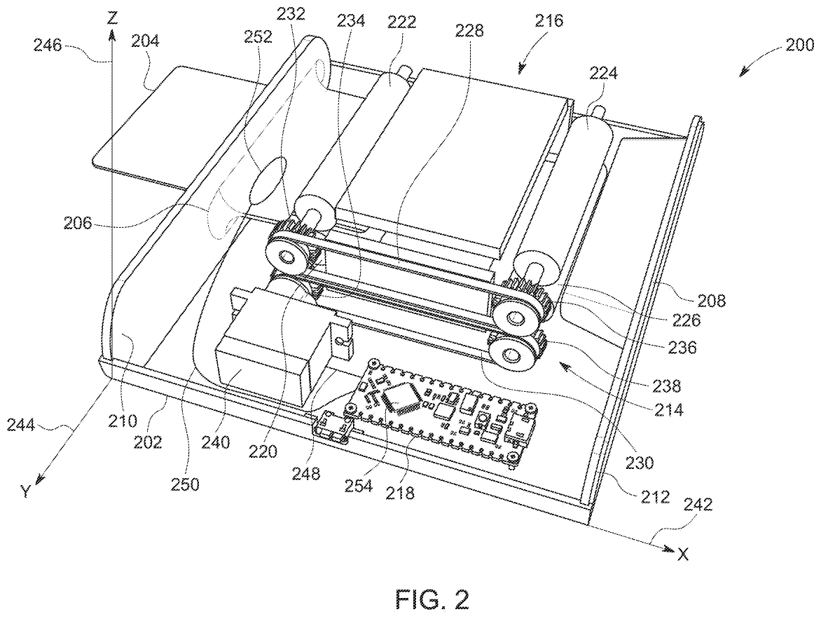

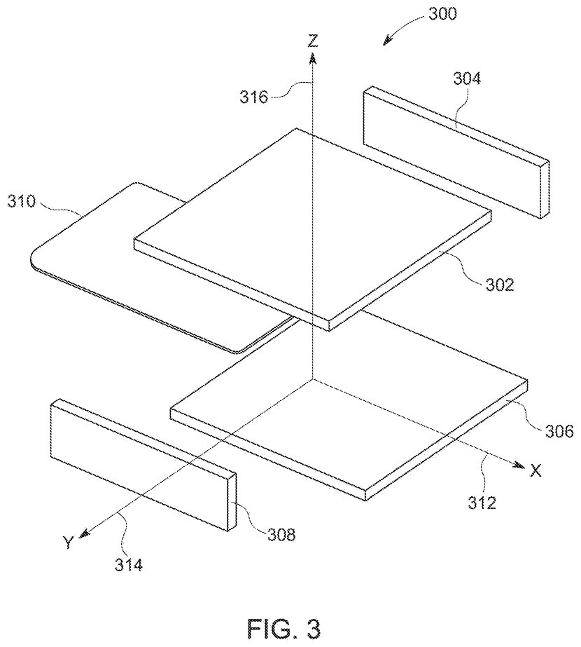

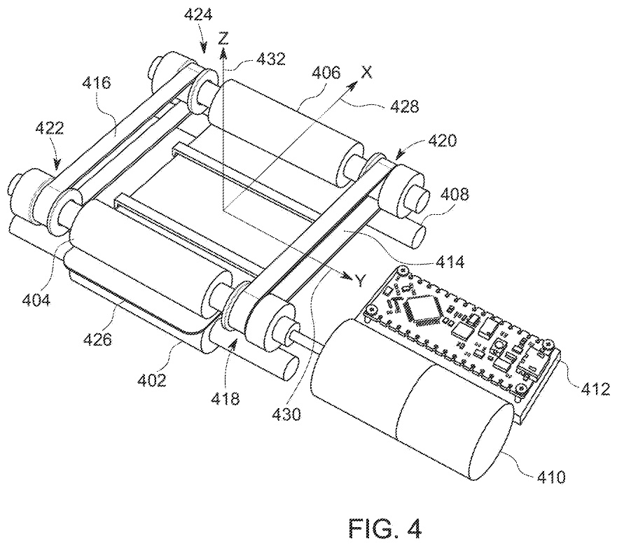

FIG. 1A depicts a perspective view of an ultraviolet germicidal irradiation device 100 according to a preferred embodiment. The device includes a substantially cubic housing 102 which comprises a first side 110 having a first aperture 106 where a card 104, such as a hotel key card, may be inserted to be disinfected by an ultraviolet light source (see FIG. 2., or FIG. 3, or FIG. 4). In this embodiment, the aperture 106 has a rectangular cross section having a width 114 and a height 116. The card 104 has a rectangular cross section having a width 128 and a height 130. A conveyor (see FIG. 2 or FIG. 4) is contained within the housing 102 which operates to receive the card 104 through the first aperture 106. The conveyor moves the card 104 along an axial direction, the X axis 122 in a planarly manner, i.e., substantially in the X-Y plane defined by the X axis 122 and the Y axis 124 and perpendicular to the Z axis 126. A control device (see FIG. 2 or FIG. 4) is configured to control a linear motion of the conveyor along the axial direction 122, thereby, moving the card 104 as it is being exposed to ultraviolet radiation from the ultraviolet light source. In this preferred embodiment, the conveyor planarly moves the card 104 along the axial direction 122 and ejects it through a second aperture (see FIG. 1B) of the housing 102.

FIG. 1B depicts a perspective view of the ultraviolet germicidal irradiation device 100 of FIG. 1A. In this view, the ultraviolet germicidal irradiation device 100 is rotated 90 degrees clockwise around the Z axis 126 where a second aperture 108 through a second side 112 of the housing 102 is shown. The second aperture 108 has a rectangular cross section having a width 118 and a height 120. In this preferred embodiment, the width 114 of the first aperture 106 and the width 118 of the second aperture 108 are substantially equal to the width 128 of the card 104, and the height 116 of the first aperture 106 and the height 120 of the second aperture 108 are substantially equal to the height 106 of the card 104, so as to provide an interference fit between the card 104 and the first aperture 106, and the card 104 and the second apertures 108 as the card 104 is received and ejected through the first aperture 106 and the second aperture 108, respectively.

In an alternative embodiment, the width 114 of the first aperture 106 and the width 118 of the second aperture 108 are substantially greater than the width 128 of the card 104, and the height 116 of the first aperture 106 and the height 120 of the second aperture 108 are substantially greater than the height 106 of the card 104, so as to provide a clearance fit between the card 104 and the first aperture 106, and the card 104 and the second apertures 108 as the card 104 is received and ejected through the first aperture 106 and the second aperture 108, respectively.

In yet another alternative embodiment, the housing 102, of the ultraviolet germicidal irradiation device 100 has only one aperture 106 through its first side 110. The card 104 may be inserted to be disinfected by an ultraviolet light source (see FIG. 2_, or FIG. 3, or FIG. 4). A conveyor (see FIG. 2 or FIG. 4) is contained within the housing 102 which operates to receive the card 104 through the first aperture 106. The conveyor planarly moves the card 104 along the X axis 122 and a control device (see FIG. 2 or FIG. 4) is configured to control a linear motion of the conveyor along the axial direction 122. The card 104 is exposed to ultraviolet radiation from the ultraviolet light source and the control device reverses the linear motion of the conveyer along the axial direction 122 so as to eject the card 104 through the first aperture 106, eliminating the need for a second aperture.

FIG. 2 depicts a perspective view of an ultraviolet germicidal irradiation device 200 according to a preferred embodiment. The device 200 includes a housing 202, a conveyor 214, an ultraviolet light source 216, and a control device 218. For illustrative purposes, a top portion of the housing 202 has been removed in this view so as to expose the internal components, i.e., the conveyor 214, the ultraviolet light source 216, and the control device 218 of the ultraviolet germicidal irradiation device 200.

Similar to the ultraviolet germicidal irradiation device 100 shown in FIG. 1A, the ultraviolet germicidal irradiation device 200 includes the substantially cubic housing 202 which comprises a first side 210 having a first aperture 206 where a card 204, such as an ATM card, may be inserted to be disinfected by an ultraviolet light source 216. The conveyor 214 is disposed within the housing 202 and operates to receive the card 204 through the first aperture 206. The conveyor 214 moves the card 204 along an axial direction, i.e., the X axis 242 in a planarly manner, i.e., substantially in the X-Y plane defined by the X axis 242 and the Y axis 244 and perpendicular to the Z axis 246 The control device 218 is configured to control a linear motion of the conveyor 214 along the axial direction 242, thereby, moving the card 204 as ultraviolet radiation from the ultraviolet light source 216 illuminates the card 204. The conveyor 214 planarly moves the card 204 along the axial direction 242 and ejects it through a second aperture 208 of the housing 202.

In this preferred embodiment, the conveyor 214 comprises a first roller 220 having a first gear 234, a second roller 222 having a second gear 232, a third roller 224 having a third gear 236, a fourth roller 226 having a fourth gear 238, a first belt 228, and a motor 240 electrically coupled with the control device 218 via a line 248. The first gear 234 engages the second gear 232, the third gear 236 engages the fourth gear 238, the first belt 228 engages the second roller 222 and the third roller 224, and the motor 240 engages the first roller 220. The conveyor further includes a second belt 230 operable to engage the first roller 220 and the fourth roller 226.

In particular, the control device 218 applies a signal to the motor 240 via the line 248 to control the rotational motion of the motor 240. The motor 240 is mechanically coupled, to the first roller 220 and rotates it in a clockwise direction around the Y axis 244. The first gear 234 also rotates in the clockwise direction around the Y axis 244 and is in rotational engagement with the second gear 232 and rotates it in the counterclockwise direction around the Y axis 244. As such, the first roller 220 rotates in the clockwise direction and the second roller 222 rotates in the counterclockwise direction around the Y axis 244. The card 204 enters the first aperture 206 and is sandwiched between the first roller 220 and the second roller 222 and thus planarly moves along the axial direction, i.e., the X axis 242. The first belt 228 is in rotational engagement with the second roller 222 and the third roller 224, thereby, rotating the third roller 224 in the counterclockwise direction around the Y axis 244. The third gear 236 also rotates in the counterclockwise direction around the Y axis 244 and is in rotational engagement with the fourth gear 238 and rotates it in the clockwise direction around the Y axis 244. As such, the third roller 220 rotates in the counterclockwise direction and the fourth roller 226 rotates in the clockwise direction around the Y axis 244. The card 204 planarly moves along the X axis 242 and is sandwiched between the third roller 224 and the fourth roller 226. The card 204 is ejected through the second aperture 208.

In an alternative embodiment, the conveyor 214 has only one belt, i.e., the first belt 228, as the conveyor is fully functional without the second belt 230. However, the second belt 230 may be utilized for stability of the conveyor 214.

A sensor 252 may be utilized to detect the card 204 as it enters through the first aperture 206 and to generate a signal in response to detecting the card 206. The control device 218 is further electrically coupled with the sensor 252 via a line 250 and is configured to receive the signal from the sensor 252 and control the linear motion of the conveyor 214 along the axial direction 242 of the housing 202 according to the signal.

The control device 218 comprises a processor 254 including a programming code stored on a storage device of said processor. The control device 218 may further comprise an analog to digital convertor (ADC) and a communication module. According to a preferred embodiment, the control device 218 is a 68HC08 processor having internal flash memory available from Freescale of Austin, Tex. It is contemplated that the processor may be a combination of individual discrete or separate integrated circuits packaged in a single housing or it may be fabricated in a single integrated circuit.

FIG. 3 depicts an exploded view of an ultraviolet light source 300 according to a preferred embodiment. The ultraviolet light source 300 comprises four panels, 302, 304, 306, and 308, each having UV-C light emitting diodes (LEDs). A card 310 planarly movies along an axial direction, i.e., the X axis 312 and is disinfected by the radiation emanating from the UV-C ultraviolet light source 300 as it moves along the X axis 312. In particular, the card 310 is illuminated from the top by the panel 302, from the bottom by the panel 306, from the right side by the panel 308, and from the left side by the panel 304. In one embodiment, the ultraviolet light source 300 may be switched on and off manually. In another embodiment, the ultraviolet light source 300 may be switched on and off via a control device, such as the control device 218 shown in FIG. 2.

FIG. 4 depicts a perspective view of a conveyor and a control device 412 according to a preferred embodiment. The conveyor includes a first roller 402, a second roller 404, a third roller 406, a fourth roller 408, a first belt 414, a second belt 416 and a motor 410, electrically coupled with the control device 412. The first belt 414 engages a first end 418 of the second roller 404 and a first end 420 of the third roller 406. The motor 410 engages the second roller 404. The second belt 416 engages a second end 422 of the second roller 404 and a second end 424 of the third roller 406.

In particular, the control device 412 applies a signal to the motor 410 via a line 248 (not shown) to control the rotational motion of the motor 410. The motor 410 is mechanically coupled to the second roller 404 and rotates it in a counterclockwise direction around, the Y axis 430. A card 426 enters a first aperture (not shown) and is sandwiched between the first roller 402 and the second roller 404 and thus planarly moves along the axial direction, i.e., the X axis 428. The first belt 414 is in rotational engagement with the second roller 404 and the third roller 406, thereby, rotating the third roller 406 in the counterclockwise direction around the Y axis 430. The card 426 planarly moves along the X axis 428 and is sandwiched between the third roller 406 and the fourth roller 408. The card 426 is ejected through a second aperture (not shown).

In an alternative embodiment, the conveyor has only one belt, i.e., the first belt 414, as the conveyor is fully functional without the second belt 416. However, the second belt 416 may be utilized to provide stability to the conveyor.

The foregoing explanations, descriptions, illustrations, examples, and discussions have been set forth to assist the reader with understanding this invention and further to demonstrate the utility and novelty of it and are by no means restrictive of the scope of the invention. It is the following claims, including all equivalents, which are intended to define the scope of this invention.

What is claimed is:

1. An ultraviolet germicidal irradiation device, comprising:
(a) a substantially cubic housing having a first aperture through a first side of the housing and a second aperture through a second side of the housing;
(b) a conveyor operable to receive a card through the first aperture, to planarly move the card along an axial direction of the housing, and to eject the card through the second aperture;
(c) an ultraviolet light source operable to disinfect the card, wherein the ultraviolet light source illuminates the card within the housing as the card moves along the axial direction of the housing; and
(d) a control device, coupled with the conveyor, wherein the control device comprises a processor including a programming code stored on a storage device of said processor, and wherein the control device is configured to control a linear motion of the conveyor along the axial direction of the housing.

2. The ultraviolet germicidal irradiation device of claim 1, wherein each of the first aperture and the second aperture has a rectangular cross section.

3. The ultraviolet germicidal irradiation device of claim 2, wherein a width of the rectangular cross section of each of the first aperture and the second aperture is substantially equal to or greater than a width of a rectangular cross section of the card, and wherein a height of the rectangular cross section of each of the first aperture and the second aperture is substantially equal to or greater than a height of the rectangular cross section of the card.

4. The ultraviolet germicidal irradiation device of claim 1, wherein the conveyor comprises a first roller, a second roller, a third roller, a fourth roller, a first belt, and a motor coupled with the control device, wherein the first belt engages a first end of the second roller and a first end of the third roller, and wherein the motor engages the second roller.

5. The ultraviolet germicidal irradiation device of claim 4, wherein the conveyor further comprises a second belt operable to engage a second end of the second roller and a second end of the third roller.

6. The ultraviolet germicidal irradiation device of claim 1, wherein the conveyor comprises a first roller having a first gear, a second roller having a second gear, a third roller having a third gear, a fourth roller having a fourth gear, a first belt, and a motor coupled with the control device, wherein the first gear engages the second gear, wherein the third gear engages the fourth gear, wherein the first belt engages the second roller and the third roller, and wherein the motor engages the first roller.

7. The ultraviolet germicidal irradiation device of claim 6, wherein the conveyor further comprises a second belt operable to engage the first roller and the fourth roller.

8. The ultraviolet germicidal irradiation device of claim 1, wherein the ultraviolet light source comprises four panels having UV-C light emitting diodes (LEDs).

9. The ultraviolet germicidal irradiation device of claim 8, wherein the four panels illuminate the card from a top side, a bottom side, a left side, and a right side of the card.

10. The ultraviolet germicidal irradiation device of claim 1, wherein the control device is further coupled with ultraviolet light source and configured to at least one of turn on and turn off the ultraviolet light source.

11. The ultraviolet germicidal irradiation device of claim 1, further comprising:
(e) a sensor operable to detect the card through the first aperture and to generate a signal in response to detecting the card;
wherein the control device is further coupled with the sensor and configured to receive the signal from the sensor and control the linear motion of the conveyor along the axial direction of the housing according to the signal.

12. The ultraviolet germicidal irradiation device of claim 1, wherein the card is one of a hotel key card, an ATM card, and a credit card.

13. An ultraviolet germicidal irradiation device, comprising:
(a) a substantially cubic housing having an aperture through a side of the housing;
(b) a conveyor operable to receive a card through the aperture, to planarly move the card along an axial direction of the housing, and to eject the card through the aperture;
(c) an ultraviolet light source operable to disinfect the card, wherein the ultraviolet light source illuminates the card within the housing, as the card moves along the axial direction of the housing; and
(d) a control device, coupled with the conveyor, wherein the control device comprises a processor including a programming code stored on a storage device of said processor, and wherein the control device is configured to control a linear motion of the conveyor along the axial direction of the housing.

14. The ultraviolet germicidal irradiation device of claim 13, wherein the aperture has a rectangular cross section.

15. The ultraviolet germicidal irradiation device of claim 14, wherein a width of the rectangular cross section of the aperture is substantially equal to or greater than a width of a rectangular cross section of the card, and wherein a height of the rectangular cross section of the aperture is substantially equal to or greater than a height of the rectangular cross section of the card.

16. The ultraviolet germicidal irradiation device of claim 11, wherein the conveyor comprises a first roller, a second roller, a third roller, a fourth roller, a first belt, and a motor coupled with the control device, wherein the first belt engages a first end of the second roller and a first end of the third roller, and wherein the motor engages the second roller.

17. The ultraviolet germicidal irradiation device of claim 16, wherein the conveyor further comprises a second belt operable to engage a second end of the second roller and a second end of the third roller.

18. The ultraviolet germicidal irradiation device of claim 11, wherein the conveyor comprises a first roller having a first gear, a second roller having a second gear, a third roller having a third gear, a fourth roller having a fourth gear, a first belt, and a motor coupled with the control device, wherein the first gear engages the second gear, wherein the third gear engages the fourth gear, wherein the first belt engages the second roller and the third roller, and wherein the motor engages the first roller.

19. The ultraviolet germicidal irradiation device of claim 18, wherein the conveyor further comprises a second belt operable to engage the first roller and the fourth roller.

20. The ultraviolet germicidal irradiation device of claim 11, wherein the ultraviolet light source comprises four panels having UV-C light emitting diodes (LEDs).

21. The ultraviolet germicidal irradiation device of claim 20, wherein the four panels illuminate the card from a top side, a bottom side, a left side, and a right side of the card.

22. The ultraviolet germicidal irradiation device of claim 11, wherein the control device is further coupled with ultraviolet light source and configured to at least one of turn on and turn off the ultraviolet light source.

23. The ultraviolet germicidal irradiation device of claim 11, further comprising:
(e) a sensor operable to detect the card through the aperture and to generate a signal in response to detecting the card;
wherein the control device is further coupled with the sensor and configured to receive the signal from the sensor and control the linear motion of the conveyor along the axial direction of the housing according to the signal.

24. A method of disinfecting a card via an ultraviolet germicidal irradiation device, said ultraviolet germicidal irradiation device comprising a substantially cubic housing having a first aperture through a first side of the housing and a second aperture through a second side of the housing, a conveyor operable to receive the card through the first aperture, to planarly move the card along an axial direction of the housing, and to eject the card through the second aperture, an ultraviolet light source operable to disinfect the card, wherein the ultraviolet light source illuminates the card within the housing as the card moves along the axial direction of the housing, and a control device, coupled with the conveyor, wherein the control device comprises a processor including a programming code stored on a storage device of said processor, and wherein the control device is configured to control a linear motion of the conveyor along the axial direction of the housing, said method comprising:
(i) receiving the card through the first aperture, via the conveyor;
(ii) planarly moving the card along the axial direction of the housing, via the conveyor;
(iii) illuminating the card within the housing as the card moves along the axial direction of the housing, via the ultraviolet light source; and
(iv) controlling the linear motion of the conveyor along the axial direction of the housing, via the control device.

25. The method of claim 24, wherein the ultraviolet germicidal irradiation device further comprises a sensor operable to detect the card through the aperture and to generate a signal in response to detecting the card and wherein the control device is further coupled with the sensor and configured to receive the signal from the sensor and control the linear motion of the conveyor along the axial direction of the housing according to the signal, said method further comprising:
(v) detecting the card through the aperture, via the sensor;
(vi) generating a signal in response to detecting the card, via the sensor;
(vii) receiving the signal, via the control device; and
(viii) controlling the linear motion of the conveyor along the axial direction of the housing according to the signal, via the control device.
